(19) United States
(12) Patent Application Publication
(10) Pub. No.: US 2025/0305010 A1
(43) Pub. Date: Oct. 2, 2025

(54) LIPID COMPOSITIONS AND METHODS OF PRODUCING SAME

(71) Applicants: Wilk Technologies International Ltd., Rehovot (IL); Yissum Research Development Company of the Hebrew University of Jerusalem Ltd., Jerusalem (IL)

(72) Inventors: Tomer KEREN, Givatayim (IL); Ezra ELLA, ModiIn (IL); Nurit ARGOV-ARGAMAN, Tel-Aviv (IL)

(73) Assignees: Wilk Technologies International Ltd., Rehovot (IL); Yissum Research Development Company of the Hebrew University of Jerusalem Ltd., Jerusalem (IL)

(21) Appl. No.: 18/862,996

(22) PCT Filed: May 8, 2023
(86) PCT No.: PCT/IL2023/050467

§ 371 Date: Nov. 5, 2024

Related U.S. Application Data
(60) Provisional application No. 63/339,466, filed on May 8, 2022.

(51) Int. Cl. C12P 7/64; C12N 5/00; C12N 5/071

(52) U.S. Cl. CPC C12P 7/64; C12N 5/0018; C12N 5/0631; C12N 2501/315; C12N 2501/33; C12N 2501/38; C12N 2501/39; C12N 2501/998; C12N 2501/999

(57) ABSTRACT

Provided is a method of increasing lipid synthesis or accumulation in mammary epithelial cells (MECs) culture and optionally secretion therefrom. The method comprising contacting the MECs with an LXR agonist in the presence of a fatty acid to thereby increase lipid synthesis or accumulation and optionally secretion of lipids. Also provided is a method of producing synthetic fat globules, the method comprising: (a) isolating lipid droplets from cells in culture; (b) encapsulating the droplets with a lipid bilayer, thereby producing synthetic fat globules. Also provided are compositions produced thereby.

### RELATED APPLICATION

This application claims the benefit of priority of U.S. Provisional Patent Application No. 63/339,466 filed on 8 May 2022, the contents of which are incorporated herein by reference in their entirety.

### SEQUENCE LISTING STATEMENT

The xml Sequence Listing, entitled 96064.xml, created on May 8, 2023, comprising 28,672 bytes, submitted concurrently with the filing of this application is incorporated herein by reference.

### FIELD AND BACKGROUND OF THE INVENTION

The present invention, in some embodiments thereof, relates to lipid compositions and methods of producing same.

The global dairy market, comprising the processing and harvesting of animal milk for human consumption, reached a value of US$718.9 Billion in 2019, and is typically sourced from cow, goat, buffalo, camel and sheep. With widespread demand for dairy products and their proactive function in the global food industry, dairy plays a crucial role in the growth of the economies worldwide.

Existing dairy milk alternatives, such as soy, almond, rice, or coconut milk fall short both in flavor and in functionality; moreover, a large part of the industrial and cultural significance of dairy milk stems from its usefulness in derivative products, such as cheese, yogurt, cream, or butter. Non-dairy plant-based milks, while addressing environmental and health concerns (and while providing adequate flavor for a small segment of the population), almost universally fail to form such derivative products when subjected to the same processes used for dairy milk.

Moreover, recent report from IATP noted, that as of 2017, the 13 top dairy companies' emissions grew 11% compared with 2015, corresponding to a 32.3 million metric ton increase in greenhouse gases equivalent to the emissions that would be released by adding an extra 6.9 million cars to the road for a year.

Mammary gland epithelial cells (MECs) can be cultured to synthesize and secret milk components to a given medium. Commonly used commercial growth medium usually include amino acids, essential fatty acids and glucose or pyruvate, which are intended to provide the cells' nutritional needs for production of milk components (and milk). See for example, Nan et al. Physiol Genomics 46:268-275, 2014. Nevertheless the secretion capacity is rather low, compared with in-vivo quantities, especially that of milk protein, milk fat and lactose.

Additional background art includes:
Cohen et al. 2015 PLOS ONE 10 (3): e0121645
Cohen et al. 2017 Journal of Mammary Gland Biology and Neoplasia (2017) 22:235-249
Hadaya, O., et al. “Pistacia lentiscus extract enhances mammary epithelial cells' productivity by modulating their oxidative status.” Scientific reports 10.1 (2020): 1-16).

### SUMMARY OF THE INVENTION

According to an aspect of the invention there is provided a method of increasing lipid synthesis or accumulation in mammary epithelial cells (MECs) culture and optionally secretion therefrom, the method comprising contacting the MECs with an LXR agonist in the presence of a fatty acid to thereby increase lipid synthesis or accumulation and optionally secretion of lipids.

According to some embodiments, the fatty acid is selected from the group consisting of oleic acid, palmitic acid, linoleic acid, arachidonic acid, eicosapentaenoic acid and docosahexaenoic acid.

According to some embodiments, the fatty acid is selected from the group consisting of oleic acid and palmitic acid.

According to some embodiments, the fatty acid is oleic acid.

According to some embodiments, the contacting is in the presence of at least one of a phenolic composition, insulin, hydrocortisone, prolactin and 8-hydroxybutirate (BHBA).

According to some embodiments, the method further comprises harvesting lipids from the MECs.

According to some embodiments, the method further comprises harvesting lipids from a medium of the culture of the MECs.

According to some embodiments, the LXR agonist is GW3965 or T0901317.

According to some embodiments, the LXR agonist is 9-cis-13,14-dihydroretinoic acid.

According to some embodiments, the LXR agonist is at least one nucleic acid agent which upregulates LXR, an activator and/or effector thereof.

According to some embodiments, the nucleic acid agent encodes LXR.

According to some embodiments, the at least one nucleic acid agent encodes LXR and PRLR.

According to some embodiments, the at least one nucleic acid agent introduces a nucleic acid alteration in a genomic sequence of the LXR, an activator and/or effector thereof.

According to some embodiments, the genomic sequence is a coding sequence.

According to some embodiments, the genomic sequence is a non-coding sequence.

According to some embodiments, the nucleic acid alteration renders the LXR, the activator and/or effector thereof constitutively active.

According to some embodiments, the nucleic acid alteration renders the LXR, the activator and/or effector thereof regulated in an inductive manner.

According to some embodiments, the nucleic acid alteration renders an LXR response element (LXRE) constitutively active.

According to some embodiments, the nucleic acid alteration renders an LXR response element (LXRE) inducible.

According to some embodiments, the culture comprises the LXR agonist, fatty acid and the phenolic composition.

According to some embodiments, the phenolic composition is selected from the group consisting of flavonol, flavanol, flavone, flavanone and anthocyanidin.

According to some embodiments, the phenolic composition is selected from the group consisting of gallic acid or myricetin or derivative thereof.

According to some embodiments, the method is effected in the presence of albumin.

According to some embodiments, the albumin is bovine serum albumin (BSA).

According to some embodiments, the albumin is BSA or plant albumin.

According to some embodiments, the MECs are human or bovine MECs.

According to an aspect of the invention there is provided a composition comprising cells obtainable according to the method as described herein or secretome or a fraction of the cells or the secretome.

According to an aspect of the invention there is provided a method of producing synthetic fat globules, the method comprising:
(a) isolating lipid droplets from cells in culture;
(b) encapsulating the droplets with a lipid bilayer, thereby producing synthetic fat globules.

According to some embodiments, the cells are mammary epithelial cells (MECs).

According to some embodiments, the lipid bilayer is a cell membrane.

According to some embodiments, the cell membrane is of mammary epithelial cells (MECs).

According to some embodiments, the lipid bilayer is a synthetic membrane.

According to some embodiments, the lipid bilayer comprises peptides.

According to some embodiments, the membrane and the lipid droplet content are from different sources.

According to some embodiments, one source of the different sources comprises MECs.

According to some embodiments, the membrane and the lipid droplet content are from the same source.

According to some embodiments, the source is a mammary epithelial cell (MEC).

According to some embodiments, the method further comprises subjecting the cells to a lipid synthesis induction protocol prior to step (a).

According to some embodiments, the lipid induction protocol is as described herein.

According to an aspect of the invention there is provided a composition comprising synthetic fat globules obtainable as described herein.

According to an aspect of the invention there is provided a composition comprising synthetic fat globules which comprise a lipid droplet core coated with a lipid bilayer.

According to some embodiments, the synthetic fat globules are of a size range of 100 nm-15 μm.

According to an aspect of the invention there is provided a nutritional product comprising the composition as described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows that LXR agonist enhances a lipid metabolism pathway via activation of SREBP1 and FASN in mammary epithelial cells (MECs). The increase in the expression of lipid metabolism-related genes following overnight incubation with LXR agonist. LXR1 and LXR2 are two different LXR agonists, as specified in the Examples section. IM and GM are controls for induction media and growth media respectively.

FIG. 2 is an image presentation showing that LXRa ameliorates the negative effect of oleic acid (OA) treatment on cells. Demonstration of the effect of LXRa treatment (1 μM/ml, TO901317) on cells viability and morphology.

FIG. 3 is a graphic presentation showing that delta triglycerides (TGs) levels inside cells are similar with or without LXRa.

FIG. 4 shows the effect of treatment with different combinations of fatty acids on lipid profile as obtained by chromatographic profiles (HPLC) of extracted lipid samples. Cells were treated with an induction step, and then with palmitic or oleic acid. Green: Bovine's milk control. Black: induction without treatment control. Blue: Treatment with oleic acid. Pink: treatment with palmitic acid. Brown: infant formula.

FIG. 5 shows spheroids in suspension. The cells produce a structure of a round ball, 15 empty from the inside, allowing polarization of the cells.

FIG. 6 shows that Mammary Epithelial Cells (MECs) can be grown on micro-beads in suspension. The cells adhere to the micro-bead surface, and the micro-bead is floating in suspension, allowing the usage of liquid bioreactor for cultivation of high cell mass.

### DESCRIPTION OF SPECIFIC EMBODIMENTS OF THE INVENTION

The present invention, in some embodiments thereof, relates to lipid compositions and methods of producing same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Mammary gland epithelial cells can be cultured to synthesize and secret milk components to the medium. Commonly used commercial growth media usually include amino acids, essential fatty acids and glucose or pyruvate, which are intended to provide the cellular nutritional needs for production of milk components. Nevertheless the secretion capacity is rather low, compared with in vivo production and secretion, especially that of milk fat.

Whilst conceiving embodiments of the invention and reducing them to practice the present inventors have identified a synergistic combination which enhances the production and even the secretion of milk lipids, i.e., triglycerides.

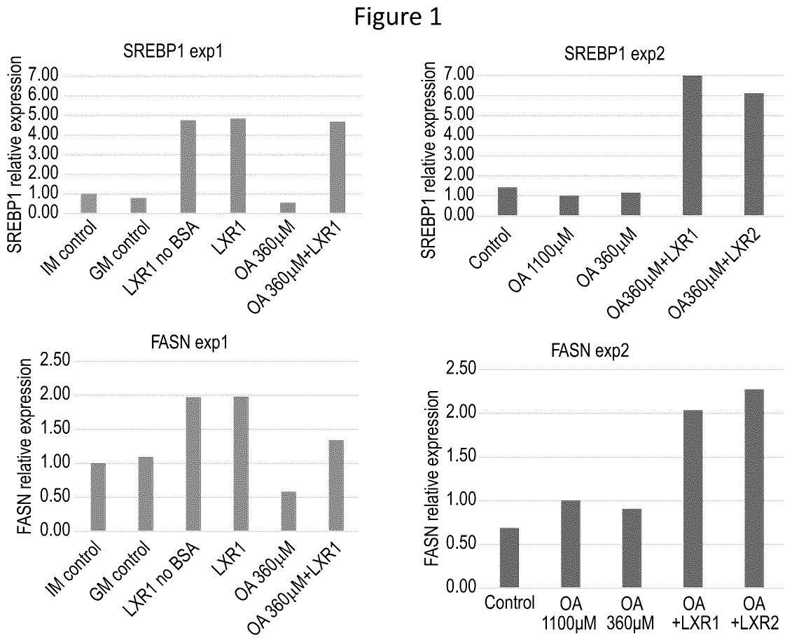

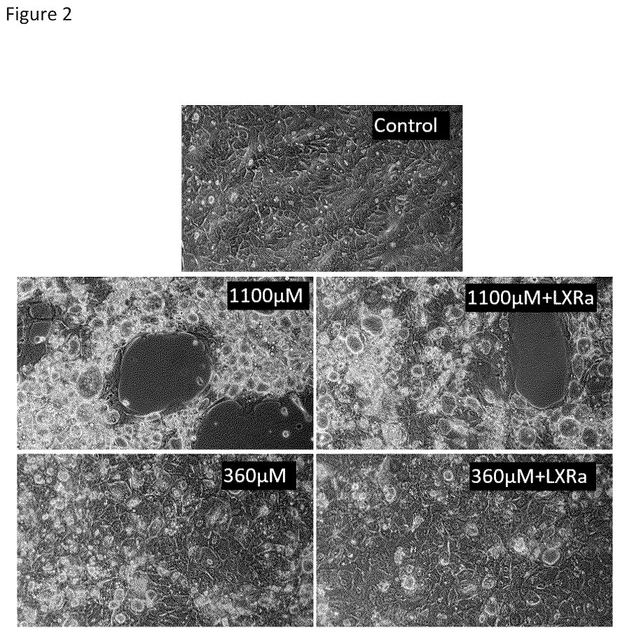

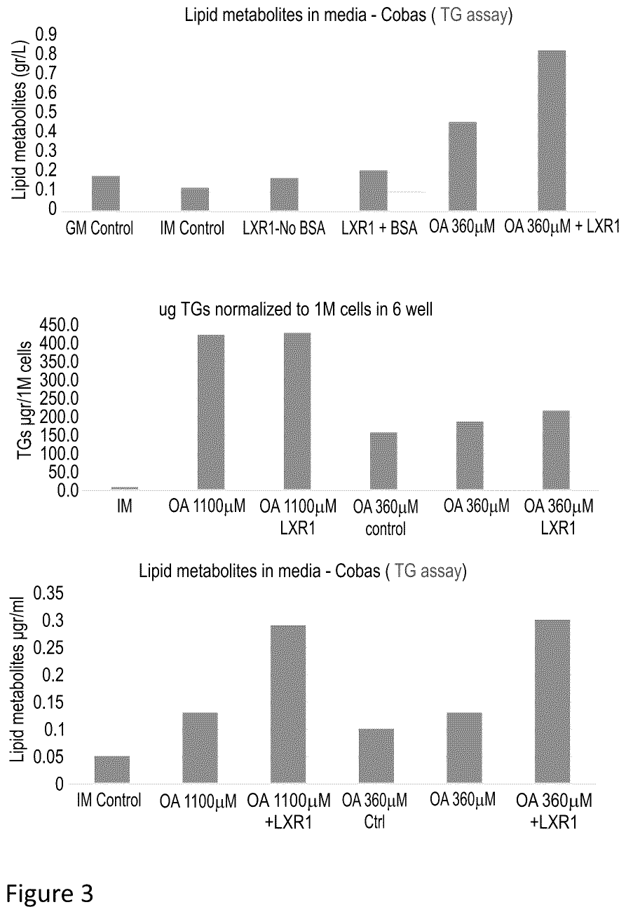

Specifically, the present inventors have found that LXR agonists can induce lipid metabolism via expression of LXR effectors, SREBP1 and FASN, in mammary epithelial cells (MECs). Activation of this pathway was evident in the absence of a fatty acid such as an oleic acid (OA), see FIG. 1. However, LXR agonists could diminish the deleterious effects of fatty acids such as high dose OA, a lipogenic substance per se, which typically lead to cell death (see FIG. 2). Overall, LXR agonists together with fatty acids increase the ability of MECs to accumulate and secrete lipids (see FIG. 3), rendering this combination valuable for producing milk components in culture.

Thus, according to an aspect of the invention there is provided a method of increasing lipid synthesis or accumulation in mammary epithelial cells (MECs) culture and optionally secretion therefrom, the method comprising contacting the MECs with an LXR agonist in the presence of a fatty acid to thereby increase lipid synthesis or accumulation and optionally secretion of lipids.

According to an additional or an alternative aspect of the invention there is provided a method of increasing lipid synthesis or accumulation in mammary epithelial cells (MECs) culture and optionally secretion therefrom, the method comprising contacting the MECs with an LXR agonist in the presence of at least one of a fatty acid and a phenolic composition (meaning in the presence of a fatty acid and/or a phenolic composition) to thereby increase lipid synthesis or accumulation and optionally secretion of lipids.

According to an additional or an alternative aspect of the invention there is provided a method of increasing lipid synthesis or accumulation in mammary epithelial cells (MECs) culture and optionally secretion therefrom, the method comprising contacting the MECs with an LXR agonist in the presence of at least one of an oleic acid (OA) and a phenolic composition (meaning in the presence of a fatty acid and/or a phenolic composition) to thereby increase lipid synthesis or accumulation and optionally secretion of lipids.

As used herein “increasing” or “increase” refers to an increase in lipid synthesis (de-novo), accumulation and/or secretion of at least 10%, 20%, 30%, 40%, 50%, 70%, 90% or more, say 1.2 fold, 1.5 fold, 2 fold, 5 fold, 10 fold or more as compared to same in MECs which are cultured under control conditions i.e., not subjected to the agent, i.e., the LXR agonist and at least one of the fatty acids such as an oleic acid (OA) and a phenolic composition (meaning in the presence of a fatty acid and/or a phenolic composition), but otherwise same conditions.

As used herein “lipid accumulation” refers to any milk lipid such as triglycerides, polar lipids, glycerophospholipids, sphingolipids, ceramide, gangliosides, cholesterol, lysophosphatidylcholine, lysophospholipids and/or free fatty acids. According to a specific embodiment, the lipid accumulation is mainly due to TG accumulation by the TG synthesis pathway.

The present teachings lead to increased lipid synthesis which can be accumulated in the cells and even secreted from the cells.

As used herein “secretion” refers to secretion of the desired component, e.g., lipids, to the medium of the culture. In some instances, this may be referred to as a “conditioned medium”. However, in other instances, the nutrient medium on which the cells feed upon (are cultured in) is separated from the medium to which the cells secrete the milk component so as to allow harvesting of the milk secretion (secretome), as further described hereinbelow.

As was previously published (see e.g., Cohen et al. PLOS One. 2015; 10 (3): c0121645) and reiterated herein, OA can increase lipid synthesis in MECs, however the effect of OA is deleterious to the cells, leading to cell death. LXR agonists inhibit the cytotoxic effect of OA possibly by allowing dispersion of lipid droplets in the cells and alternatively or additionally allowing secretion of the accumulated lipids from the cells.

As used herein “tissue culture” refers to ex vivo growth of cells.

The cells can be isolated single cells, cell clumps (e.g., organoids) or comprised in a tissue which typically comprises more than one cell type, e.g., mammary epithelial cells and mammary stromal cells (mainly fibroblast and progenitor cells of the mammary gland).

According to a specific embodiment, the methods and systems described herein are “generally recognized as safe” (GRAS).

As used herein “mammary epithelial cells (MECs)” refers to a luminal, basal and alveolar cells. Typical markers include cytokeratin 18, cytokeratin 14, EpCAM, progesterone receptor, estrogen receptor, prolactin receptor, Elf5 and CD24.

The mammary cell can be of a human being or any mammal but preferably a dairy animal such as cattle (cows), buffalos, goats, sheep, and camels, as well as less commonly used such as yak, water buffalo, horses, donkeys, or even reindeer. For research use also contemplated herein are mice and rats.

According to a specific embodiment, the MECs are of a bovine or human source.

The cells can be primary cells or a cell line such as an immortalized cell line e.g., genetically transformed/infected to express an immortalizing gene, e.g., MCF12A or TERT.

For example, mammary epithelial cells can be obtained from surgical explants of dissected mammary tissue (e.g., breast, udder, teat). Generally, after surgical dissection of the mammary tissue, any fatty or stromal tissue is manually removed under aseptic conditions, and the remaining tissue of the mammary gland is enzymatically digested with collagenase and/or hyaluronidase prepared in a chemically defined nutrient media, which is typically composed of ingredients that are GRAS. The sample is maintained at 37° C. with gentle agitation. After digestion, a suspension of single cells or organoids is collected, either by centrifugation or by pouring the sample through a sterile nylon cell strainer. The cell suspension is then transferred to a tissue culture plate coated with appropriate extracellular matrix components (e.g., collagen, laminin, fibronectin).

Alternatively, explant specimens can be processed into small pieces, for example by mincing with a sterile scalpel. The tissue pieces are plated onto a suitable surface such as a gelatin sponge or a plastic tissue culture plate coated with appropriate extracellular matrix.

The plated cells are maintained at 37° C. in a humidified incubator with an atmosphere of 5% CO₂. During incubation, the media is exchanged about every 1 to 3 days and the cells are sub-cultured until a sufficient viable cell number is achieved for subsequent processing, which may include preparation for storage in liquid nitrogen; development of immortalized cell lines through the stable transfection of genes such as SV40, TERT (as mentioned above), or other genes associated with senescence; isolation of mammary epithelial, myoepithelial, and stem/progenitor cell types by, for example, fluorescence-activated cell sorting.

For example, the present inventors used a primary culture of bovine mammary epithelial cells isolated from lactating Holstein cows according to an established protocol (Cohen et al., 2015, supra).

Specifically, a primary culture of MECs is isolated from mammary biopsies. Briefly, the tissue is collected from lactating cows and immediately submerged in ice-cold growth medium with antibiotics and heparin supplementation to prevent cell clotting. The tissue is minced and digested by shaking in a growth medium supplemented with enzymes such as collagenases and hyaluronidases. After incubation, the suspension is filtered through a mesh (e.g., 250 μm) and the filtrate is centrifuged. The sediment is treated with trypsin-EDTA and DNase. The cells are then washed with growth medium supplemented with heparin and treated with DNase alone, filtered through a cell strainer and washed with the growth medium.

According to some embodiments of the invention, MECs are allowed to proliferate, typically to at least 80% of confluence in 2D settings or to at least about 1×10⁶ cells per ml in suspension settings. According to some embodiments, this is followed by an induction (also termed differentiation or maturation) step.

Hence, according to some embodiment, MECs are grown in culture supplemented with insulin, hydrocortisone and/or prolactin that allow maturation, i.e., secretion of milk components to the culture medium.

According to a specific embodiment, induction is done when primary cells (e.g., MECs) or ex-vivo differentiated pluripotent stem cells (ESCs or iPSCs) are used.

According to a specific embodiment, the method does not include an induction step when immortalized cells are used.

According to a specific embodiment, prolactin is added at the induction step. According to a specific embodiment, for cell proliferation the medium is devoid of prolactin. In other embodiments, prolactin is present at both expansion and induction steps, albeit same amount or higher amounts are used at the induction step as compared to the proliferation (expansion) step.

Following are some exemplary non-limiting MECs which can be used according to the present teachings.

Immortalized human breast epithelial cells-SV40 is an immortalized breast epithelial cell line, which is not tumorigenic and does not show anchorage-independent growth. Cells in the G1, S, and G2/M phases are normally distributed, and they express normal breast epithelial markers (E-cadherin, CK7/18 and CK5/14).

Bovine mammary epithelial cell cultures are described in Jedrzejczak et al. In Vitro Cell Dev Biol Anim. 2014; 50 (5): 389-398.

HC11 mammary epithelium cell line is available from the ATCC.

Anand et al. describes the establishment and characterization of a Buffalo (Bubalus bubalis) mammary epithelial cell line www (dot) doi (dot) org/10 (dot) 1371/journal (dot) ponc (dot) 0040469.

According to some embodiments of the invention, the MECs comprise ex vivo differentiated MECs. Differentiation to MECs can be from a stem cell, e.g., pluripotent stem cells or induced pluripotent stem (iPS) cells.

Hassiotou et al. Stem Cells. 2012 October;30 (10):2164-74. doi: 10.1002/stem.1188. show that human breastmilk contains stem cells (hBSCs) with multilineage properties. Breastmilk cells from different donors displayed variable expression of pluripotency genes normally found in human embryonic stem cells (hESCs). These genes included the transcription factors (TFs) OCT4, SOX2, NANOG, known to constitute the core self-renewal circuitry of hESCs. The methodology described in this publication represents a protocol to generate human mammary like cells and/or organoids from hBSCs.

WO2021219634 and WO2021219635 each describe methods of differentiation and culturing mammary epithelial cells in suspension. For example by: i) culturing hBSCs in an appropriate culture medium (for example MammoCult medium, optionally supplemented with antibiotic-antimycotic solution and fungizone) and after one week collecting mammospheres formed thereof; and ii) growing such mammospheres in an appropriate system (such as a mammary differentiation medium comprising for example culture medium RPMI (Roswell Park Memorial Institute) 1640 with L-glutamine optionally supplemented with fetal bovine serum (FBS), insulin, epidermal growth factors (EGF), hydrocortisone, antibiotic-antimycotic solution and fungizone) for at least 1 week, for example 2 to 4 weeks, to generate lactocytes. In one embodiment of these publications, generating lactocytes comprises: i) aggregating and culturing hBSCs in an appropriate culture medium (for example MammoCult medium) in non-adherent conditions for mammospheres formation; and ii) growing such mammospheres in a 3D appropriate system (for example a mixed floating gel composed of matrix protein such as Matrigel and/or Collagen or in suspension cultures in non-adherent plates) for at least 10 days to generate lactocytes. In one embodiment, mammary commitment is obtained by applying a conditioned medium (for example EpiCultB) supplemented with specific factors (for example Parathyroid hormone (pTHrP), hydrocortisone, insulin, FGF10, and HGF). Generation of mammary-like organoids can be done by culturing the cells under conditions selected from the group consisting of: 2D monolayers of cells, 2D with attached EBs, in suspension in non-adherent plates and in mixed floating gel. Additional protocols for the generation of monolayers and organoids are provided in these publications and are incorporated by reference.

According to some embodiments, the MECs comprise a MEC monolayer. A cell monolayer is typically grown under adherent conditions, using a substrate adherent matrix or a feeder layer (stroma). However, suspension cultures are also contemplated herein (e.g., mammospheres).

Examples 5 and 6 below show the growth of MECs as spheres (without any scaffold) or on beads (i.e., scaffold).

Additional examples for growth in suspension include, but are not limited to Soboleaska et al. European Journal of Cell Biology 90 (2011) 854-864; and Qu et al. PLOS ONE I DOI: 10.1371/journal.pone.0131285, each of which is incorporated by reference in its entirety.

It may be considered beneficial to grow the cells as a monolayer to achieve a polarity which enables nutrition of the cells from one position and collection of the secretome from another, i.e., a polar system, see e.g., WO2021142241, further described hereinbelow.

The cells can be native or transgenic cells (see Kuan et al. infra) such as modified to express a recombinant protein, e.g., antibodies.

Regardless of the method used, once MECs are obtained they are admixed with (contacted or incubated with) a medium composition.

As used herein “medium” refers to an artificial or synthetic medium with a defined chemical structure.

Commonly used commercial growth media usually include amino acids, essential fatty acids and glucose or pyruvate, which are intended to provide the cellular nutritional needs for production of milk components.

The cells can be expanded in vitro (ex-vivo) until enough cells are obtained.

According to a specific embodiment, the cells are incubated in an induction medium which is aimed to direct cells toward differentiation and production of milk components (e.g., lipids, carbohydrates and/or proteins).

The medium can be supplemented with lactogenic factors for example prolactin, hydrocortisone, and insulin.

For example, the medium can be a basal medium (e.g., DMEM/F12) or complex medium (e.g., RPMI-1640, IMDM) supplemented with bovine serum albumin (BSA e.g., 0.15% (v/v)) and insulin (e.g., 1 μg/ml), hydrocortisone (e.g., 0.5 μg/ml) and prolactin (e.g., 1 μg/ml) for a sufficient time, e.g., 48 h, to induce lactogenic response. Exemplary ranges are provided infra: BSA (0.001-5%); Insulin (0.001-1 μg/ml); hydrocortisone (0.05-5 μg/ml); prolactin (0.01-10 μg/ml).

According to a specific embodiment, the medium can be plant-based media, composed of plant based recombinant hormones and albumin of plant origin. Examples of such media compositions include, but are not limited to, ExCellerate iPSC Expansion Medium, KM-acf medium (ScienCell), Cnt-Prime iPS Epithelial Differentiation Medium (Zenbio), EpiCM-A-PRF media (Sciencell) or equivalents thereof.

Regardless whether an induction step is present or not, the cells are subjected to LXR and fatty acid and/or phenolic composition to improve secretion of milk components.

As mentioned, the nutritional medium is mixed (supplemented with) a biologically effective concentration of an LXR agonist and a fatty acid.

In the context of the disclosure, the term “biologically effective amount” or “biologically effective concentration” means the amount or (weigh, volume v/v or w/v or w/w) concentration of the active agent or composition needed to affect the desired biological, often beneficial, result. The amount of agent employed in the medium will be that amount necessary to deliver a biologically effective amount of the agent to achieve the desired biologic result.

As used herein “at least one” refers to 1, 2, 3, 4, 5 or more, but not more than 10 agents.

According to some embodiments of the invention, the agent promotes accumulation and/or secretion of lipids that may be referred to as a “lactogenic response”.

As used herein “a fatty acid” refers to Fatty acids (FAs) are one of the most important substances in milk, crucial for the correct development of the child in the prenatal, postnatal and infant stages. FAs play a vital role in energy processes taking place in cells, are the principal building material of cell membranes, and are precursors of important metabolic compounds.

In milk, most of the unsaturated fatty acids are MUFA, and oleic acid (18:1) being the most important one. Long chain polyunsaturated fatty acids (LCPUFA), including essential fatty acids (EFAs) such as linoleic acid (C18: 2 n-6) and α-linolenic acid (C18: 3 n-3), are also crucial for proper development of the nervous system, retina, and other structures. LCPUFA form biologically important long chain polyunsaturated derivatives such as γ-Linolenic acid (C18: 3 n-6), Arachidonic acid (C20: 4 n-6), Eicosapentaenoic acid (C20: 5 n-3), Omega-3. and Docosahexaenoic acid (C22: 6 n-3).

According to a specific embodiment the fatty acid belong to the long-chain fatty acids (LCFA) having aliphatic tails of 13 to 33 carbons.

According to a specific embodiment, the fatty acid is selected from the group consisting of oleic acid, palmitic acid, linoleic acid, arachidonic acid, cicosapentaenoic acid and docosahexaenoic acid.

According to a specific embodiment, the fatty acid is selected from the group consisting of oleic acid and palmitic acid.

According to a specific embodiment, the fatty acid is oleic acid.

The term refers to one or more (e.g., 2-5, 2-3, 2-4) fatty acids.

As used herein “oleic acid” refers to the fatty acid having the lipid number 18:1 cis-9 lipid. Oleic acid is commercially available from Merck and Avanti. Food grade oleic acid is available from Univar solutions.

The effect of a fatty acid on accumulation or secretion of fat can be determined as described hereinbelow in the Examples section or specifically, using, the Cobas assay for determining triglycerides (TGs) by indirect glycerol content in the medium or another assay which determines TGs in the cells lysate. Specific fluorescence and bright field microscopy visualization of TG accumulating lipid droplets can be performed by using Nile red and Oil Red staining respectively.

For instance, in order to analyze lipid secretion, total lipids are extracted from the collected medium. When intracellular lipids are analyzed the same can be done on lysed cells. Though it will be appreciated that the intracellular lipid composition may be different from the secreted lipids. The medium or the cells are diluted in Folch reagent (Chloroform-Methanol 2:1). After overnight incubation with cold extraction at 4° C., the upper phase is removed, and the lower phase is filtered through glass wool. Samples are then evaporated under a nitrogen stream at 65° C., diluted in chloroform:methanol (97:3, v/v) and stored at −20° C. until injection for HPLC analysis. Separation of polar and neutral lipids is performed on a silica column using with an evaporative light-scattering detector. The specific running conditions are described in the literature or in the Examples section which follows. The separated lipids are identified using external standards. Quantification can be performed against external standard curves and expressed as μg/per 10⁶ live cells or as weight % out of the sum of phospholipids (μg) in the sample. Live cell number can be determined with a hemocytometer using trypan blue staining.

According to a specific embodiment, the effective concentration of the fatty acid is about, 50-1000 μM, 100-1000 μM, 100-500 μM, 50-500 μM or 50-360 μM.

According to a specific embodiment, the effective concentration of oleic acid is about, 50-1000 μM, 100-1000 μM, 100-500 μM, 50-500 μM or 50-360 μM.

To reduce the toxic effects of the fatty acid, e.g., oleic acid, it is preferably provided in the presence of albumin such as BSA or plant albumin. Albumin protects against the deleterious effects of high-level the fatty acid, e.g., oleic acid e.g., above 100 μM, such as cytotoxicity, oxidative stress, apoptosis and necrosis. Examples of plant albumins are well known in the art, e.g., FuturMeat chickpea and any other 2S albumin, see e.g., doi: 10.1016/j.ijbiomac.2020.09.04, which is hereby incorporated by reference in its entirety.

Thus, according to some embodiments of the invention, a concentration of the albumin (e.g., BSA or plant based) is between about 0.5-32 mg BSA/ml medium when provided with the fatty acid, e.g., oleic acid.

According to other embodiments, the biologically effective concentration of the at least one of: the fatty acid, e.g., oleic acid is between about 50 M and about 360 M, and the albumin (e.g., BSA or plant albumin) concentration is between about 0.5 mg albumin (e.g., BSA or plant albumin)/ml Medium and about 32 mg/ml.

Liver X receptor, also termed as “LXR” is a member of the nuclear receptor family of transcription factors and is closely related to nuclear receptors such as the PPARs, FXR and RXR. Liver X receptors (LXRs) are important regulators of cholesterol, fatty acid, and glucose homeostasis. Endogenous oxysterols act as their ligands and as such are physiological agonists.

Two isoforms of LXR have been identified and are referred to as LXRa and LXRB. The liver X receptors are classified into subfamily 1 (thyroid hormone receptor-like) of the nuclear receptor superfamily, and are given the nuclear receptor nomenclature symbols NR1H3 (LXRa) and NR1H2 (LXRB) respectively, activated by each of the following agonists: 25-hydroxycholesterol, GW3965 and TO901317.

Liver X receptor alpha is also referred to herein as an abbreviation for an LXR agonist.

According to a specific embodiment, the activity of LXR is in eliciting target gene expression such as of ABC, ApoE, CETP, ChREBP, CYP7A1, FAS, LPL, LXRa, LXRb, SREBP-1c or specifically as analyzed in the Examples section:

FASN—(Type I Fatty Acid Synthase) a member of the FAS family.

SREBP-1c-Sterol Regulatory Element Binding Protein 1c or any of ABCA1, ABCG1, LPCAT3, Arg1, Arg2, IL-10

Methods of analyzing gene expression (e.g., increase in gene expression) include, but are not limited to, analyzing gene expression at the level of mRNA (e.g., RT-PCR, Northern blot and the like) or at the level of protein (e.g., Western blot, ELISA and the like).

Agonists of LXR may act as their physiological ligands.

Examples include, but are not limited, to endogenous oxysterols.

Examples of oxysterols that can be used include, but are not limited to, as 22 (R)-hydroxycholesterol, 24 (S)-hydroxycholesterol, 27-hydroxycholesterol or cholestenoic acid.

Synthetic LXR agonists are well known in the art, and include, but are not limited to, GW3965 and T0901317 (also termed herein as LXR1 and LXR2, respectively in the Examples section which follows), each available from commercial vendors e.g., Sigma. Other agonists include, but are not limited to 25-hydroxycholesterol, SR9238, IMB-808, SR9243, LG100754, Ethoxycarbonylmethyl, DMHCA, Tricthyl 2-phosphonobutyrate, Indole-7-carboxaldehyde, desmosterol (D6) ester, SR1078, 27-hydroxycholesterol, GSK4112, 27-hydroxycholesterol-d6.

According to a specific embodiment, the compound is a part of a cluster of vitamin A, along with the precursors 9-cis retinol, 9-cis retinyl esters, 9-cis-13,14-dihydroretinol, 9-cis-13,14-dihydroretinyl esters, which activate LXR signaling by binding to the LXR-RXR heterodimer. Hence According to some embodiments, the compound can be vitamin A or derivatives, a precursor 9-cis retinol, 9-cis retinyl esters, 9-cis-13,14-dihydroretinol or 9-cis-13,14-dihydroretinyl ester.

According to some embodiments, the LXR agonist is 9-cis-13,14-dihydroretinoic acid.

According to some embodiments, the LXR agonist is at least one nucleic acid agent which upregulates LXR, an activator and/or effector thereof.

According to some embodiments, the nucleic acid agent encodes LXR.

According to some embodiments, the at least one nucleic acid agent encodes LXR and PRLR which are shown to have a synergic effect on lipid synthesis or secretion.

According to some embodiments, the at least one nucleic acid agent introduces a nucleic acid alteration in a genomic sequence of said LXR, an activator and/or effector thereof.

According to some embodiments, the genomic sequence is in a a coding sequence. For example, such as in LXR alpha e.g., H383E, LXR E387Q, LXR H390E (Bedi et al.: Nucl Receptor Res. 2017; 4:101302).

According to some embodiments, the genomic sequence is a non-coding sequence.

Association research of LXRa and LXRb SNPs analysis revealed that LXR activity is affected by SNPs in the non-coding region as rs 12221497 are associated higher triglycerides levels (see e.g., Zhang et al. Steroids Volume 185, September 2022, 109057).

According to some embodiments, the nucleic acid alteration renders said LXR, said activator and/or effector thereof constitutively active.

According to some embodiments, the nucleic acid alteration renders said LXR, said activator and/or effector thereof regulated in an inductive manner.

According to some embodiments, the nucleic acid alteration renders an LXR response element (LXRE) constitutively active.

According to another embodiment, constitutive activation is achieved by removing LXRE and SER repression by Fox01 (Ganti et al. PNAS 114 (6) E951-E960)

According to some embodiments, the nucleic acid alteration renders an LXR response element (LXRE) inducible. such as VP16-LXRa (e.g., Hong et al. J Lipid Res. 2011 March; 52 (3): 531-539).

According to some embodiments, the LXR agonist is at least one nucleic acid agent which upregulates, LXR, an activator and/or effector thereof, collectively termed as members of the LXR pathway.

According to a specific embodiment, the STAT5 pathway activates LXR. Stat5 is a transcription factor that plays a role in the regulation of several biological processes, including cell proliferation, differentiation, and survival. It has been shown to activate LXRa through direct binding to an enhancer region in the LXRa promoter. According to a specific embodiment, the activator is a component of the Stat5 pathway.

This activation of LXRa by Stat5 results in increased expression of genes involved in lipid metabolism and cholesterol transport, including ABCA1 and ABCG1. The mechanism by which Stat5 activates LXRa is thought to involve the recruitment of co-activators and chromatin remodeling complexes to the promoter region, resulting in increased transcriptional activity. It is also possible that Stat5 activate RXR expression, and that Degradation of FoxO1 by Akt activation removes the repression from LXRE & SRE elements (www (dot) pubs (dot) acs (dot) org/doi/10.1021/acs.jafc.0c05237).

According to some embodiments of the invention, the LXR effector genes include, but are not limited to ABC, ApoE, CETP, ChREBP, CYP7A1, FAS, LPL, LXRa, LXRb, SREBP-1c.

According to such embodiments, contacting involves transformation, infection or transduction protocols.

Upregulation of at least one nucleic acid agent which upregulates LXR, an activator and/or effector thereof can be effected at the genomic level (i.e., activation of transcription via promoters, enhancers, regulatory elements), at the transcript level (i.e., correct splicing, polyadenylation, activation of translation) or at the protein level (i.e., post-translational modifications, interaction with substrates and the like).

Following is a list of agents capable of upregulating the expression level and/or activity of at least one nucleic acid agent which upregulates LXR, an activator and/or effector thereof.

An agent capable of upregulating expression of at least one nucleic acid agent which upregulates LXR, an activator and/or effector thereof may be an exogenous polynucleotide sequence designed and constructed to express at least a functional portion of the LXR, an activator and/or effector thereof. Accordingly, the exogenous polynucleotide sequence may be a DNA or RNA sequence encoding at least one nucleic acid agent which upregulates LXR, an activator and/or effector thereof molecule, capable of activating the LXR pathway.

The phrase “functional portion” as used herein refers to part of the LXR, an activator and/or effector thereof protein (i.e., a polypeptide) which exhibits functional properties of the enzyme such as binding to a substrate. According to preferred embodiments of some embodiments of the invention, the functional portion of LXR, an activator and/or effector thereof protein is a polypeptide sequence or a DNA or RNA encoding same.

LXR, an activator and/or effector thereof protein have been cloned from mammals, e.g., dairy producing mammals or humans. Thus, coding sequences information for LXR, an activator and/or effector thereof protein is available from several databases including the GenBank database available through www (dot) ncbi (dot) nlm (dot) nih (dot) gov/.

To express exogenous LXR, an activator and/or effector thereof protein in mammalian cells, a polynucleotide sequence encoding a LXR, an activator and/or effector thereof protein is preferably ligated into a nucleic acid construct suitable for mammalian cell expression. Such a nucleic acid construct includes a promoter sequence for directing transcription of the polynucleotide sequence in the cell in a constitutive or inducible manner.

It will be appreciated that the nucleic acid construct of some embodiments of the invention can also utilize LXR, an activator and/or effector thereof protein homologues which exhibit the desired activity (i.e., increase lipid accumulation or secretion). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to for instance the human or bovine sequences of LXR, an activator and/or effector thereof protein, DNA or mRNA encoding same, as determined using the BestFit software of the Wisconsin sequence analysis package, utilizing the Smith and Waterman algorithm, where gap weight equals 50, length weight equals 3, average match equals 10 and average mismatch equals −9.

Constitutive promoters suitable for use with some embodiments of the invention are promoter sequences which are active under most environmental conditions and most types of cells such as the cytomegalovirus (CMV) and Rous sarcoma virus (RSV). Inducible promoters suitable for use with some embodiments of the invention include for the tetracycline-inducible promoter (Zabala M, et al., Cancer Res. 2004, 64 (8): 2799-804).

According to a specific embodiment, the upregulation is of more than one gene in the LXR pathway. As shown in Example 4, a synergic effect on TAGs metabolism was observed when LXR and PRLR were overexpressed in human mammary epithelial cells from mammary gland.

LXR upregulation can be added any time before fatty acid treatment as preparation of the cells, or concomitantly with the fatty acid. According to some embodiment, the fatty acid is added after cells reach a predetermined level of confluence, e.g., above, 50%, 60%, 70%, 80%, 90% or 95%,

Typical culturing period with LXR agonists (e.g., small molecules) and the fatty acid, e.g., oleic acid can be in the range of 20 min to 36 h, e.g., 12-24 hours. The phenolic composition can be added at the timing of adding the fatty acid in addition to the fatty acid or as an alternative to its presence.

When the LXR is a nucleic acid agent and a constitutive expression is contemplated, the typical culturing period with the phenolic compositions or the fatty acid, e.g., oleic acid can be in the range of 20 min to 36 h, e.g., 12-24 h.

The culture may be supplemented with a phenolic composition which may increase also lipid accumulation (see Shalev et al. Heliyon Volume 8, Issue 3, March 2022, e09025).

As used herein “a phenolic composition” refers to a non-toxic (to consumption by human or domesticated animal) chemical which comprises at least one phenol group.

According to a specific embodiment, the phenolic composition is a derivate of gallic acid, or a glycosylated form of gallic acid or a flavanol, flavone, flavonoid, anthocyanin, or compounds known as hydrolysable or condensed tannin. Such compounds include polymers of flavonoids of high molecular weight and polyesters of gallic or ellagic acid that are bound to different sugars. According to a specific embodiment, the composition is a polyphenol.

As used herein “polyphenols” refers to a family of organic compounds characterized by multiples of phenol groups, which are typically naturally occurring in plants.

According to a specific embodiment, the phenolic composition is selected from the group consisting of a flavonol, flavanol, flavone, flavanone and an anthocyanidin.

According to a specific embodiment, the phenolic composition is glycosylated.

Exemplary phenolic compositions which can be used in accordance with the present teachings, include, but are not limited to:

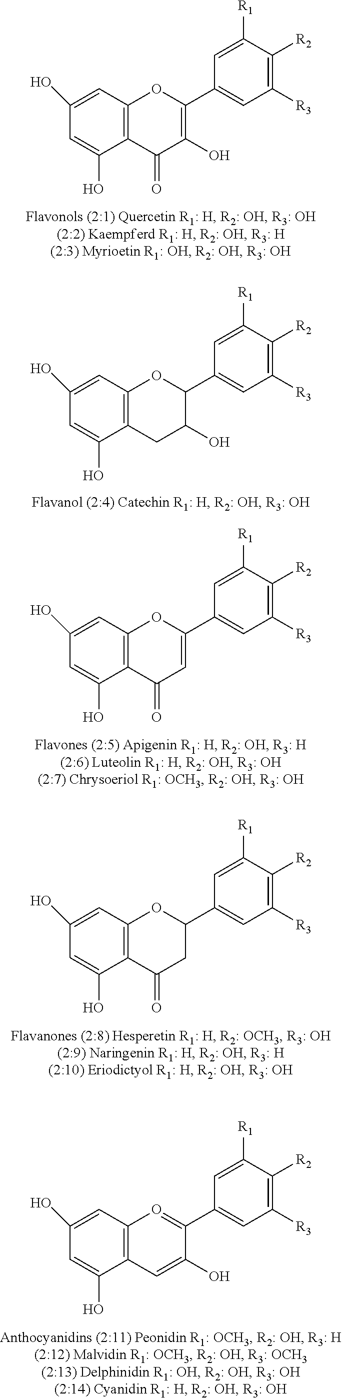

According to a specific embodiment, the phenolic composition is oleuropein, chlorogenic acid, catechin or myricetin such as available from Cayman Chemicals (Ann Arbor, MI, USA). According to a specific embodiment, the phenolic composition is a galloyl derivative, favonol glucoside or catechin.

According to a specific embodiment, the phenolic composition is a dietary phenolic compound typically originating from a plant derived food source, typically rich in secondary metabolites.

According to a specific embodiment, the phenolic composition is synthetic.

According to a specific embodiment, the phenolic composition is purified from a natural source, e.g., plant.

According to a specific embodiment, the phenolic composition is comprised in a plant extract, e.g., an ethanol extract, a chloroform extract, a hexane extract an ethyl acetate extract. According to a specific embodiment, the plant extract is a chloroform extract or a hexane extract.

Examples of plants from which the phenolic compositions can be extracted/purified include, but are not limited to Pistacia lentiscus, Buckwheat, Japanese pagoda tree, Eucalyptus, Quebrachol.

According to a specific embodiment, the phenolic composition is gallic acid or derivative thereof, such as polyesters of gallic or ellagic acid that are bound to different sugars like epigallocatechin gallate (EGCG), catechin.

A combination of phenolic compositions can act in synergy in promoting secretion of milk components.

The present inventor has shown that the selection of the phenolic composition can affect the accumulated/secreted milk component.

For example, it is shown that polyphenolic compounds such as glycosylated polyphenols, e.g., rutin and myricetin or derivatives thereof, e.g., EGCG, catechin, can increase protein, lactose and triglyceride secretion, whilst gallic acid increases lipid droplet accumulation and lactose secretion.

It is suggested, without being bound by theory, that like pyruvate, phenolic compositions act as antioxidants, thus causing glucose sparing that eventually lead to oligosaccharide synthesis and secretion.

It is suggested that the composition of the milk components can be determined by the selected phenolic composition. Thus, for instance, glycosylated polyphenols such as myricetin can lead to protein, lactose and triglyceride secretion, while gallic acid, intracellular lipid accumulation, lipid secretion, and lactose secretion.

According to a specific embodiment, the phenolic composition is purified from a natural source, e.g., plant extract or fraction thereof (not a whole extract).

According to a specific embodiment, the phenolic composition is gallic acid or derivative thereof such as methyl-gallate, Ethyl-gallate, Catechin and Epi-Catechin.

According to a specific embodiment, the biologically effective concentration of the gallic acid is between about 1 ppm and 3 ppm.

Cells obtainable according to the present teachings are contemplated herein.

According to a specific embodiment, cells treated with LXRa and OA are more viable than cells treated with OA alone, such as determined by trypan blue staining.

According to a specific embodiment, the cells are not goat cells.

Culturing the MECs in the media described herein can be done using methods which are well known in the art.

For example:

Sharfstein et al. Biotechnology and Bioengineering, Vol. 40, Pp. 672-680 (1992) describes a basic method for culturing MECs in extended-batch and hollow-fiber reactor cultures. Batch cultures are performed on Costar polycarbonate membrane inserts, allowing basal and apical exposure to medium. Protein production, for instance, is induced in both batch and hollow-fiber cultures in hormone supplemented medium.

Kuan et al. J. Anim. Sci. Vol. 88, E-Suppl. 2/J. Dairy Sci. Vol. 93, E-Suppl. 1/Poult. Sci. Vol. 89, E-Suppl. 1 describe culturing of mammary gland-like structures (gland ducts, lateral buds, and alveoli). Hollow fiber bioreactors are used for large-scale mammalian cell culture to produce milk components, e.g., recombinant proteins. The hollow fibers provide a culture system with a high surface to volume ratio. The system allows efficient exchange of nutrients and waste products across the fiber wall.

WO20211142241 describes a cell culture system designed for the collection of milk. The system is designed to support compartmentalized secretion of the product (i.e., secretome) such that the milk or secretome is not exposed to the media that provides nutrients to the cells. In the body, milk-producing epithelial cells line the interior surface of the mammary gland as a continuous monolayer. The monolayer is oriented such that the basal surface is attached to an underlying basement membrane, while milk is secreted from the apical surface and stored in the luminal compartment of the gland, or alveolus, until it is removed during milking or feeding. Tight junctions along the lateral surfaces of the cells ensure a barrier between the underlying tissues and the milk located in the alveolar compartment. Therefore, in vivo, the tissue of the mammary gland is arranged such that milk secretion is compartmentalized, with the mammary epithelial cells themselves establishing the interface and maintaining the directional absorption of nutrients and secretion of milk.

According to this embodiment, a cell culture apparatus that recapitulates the compartmentalizing capability of the mammary gland that may be used to collect milk from mammary epithelial cells grown outside of the body. Such an apparatus can include a scaffold to support the proliferation of mammary cells at the interface between two compartments, such that the epithelial monolayer provides a physical boundary between the nutrient medium and the secreted milk. In addition to providing a surface for growth, the scaffold provides spatial cues that guide the polarization of the cells and ensures the directionality of absorption and secretion.

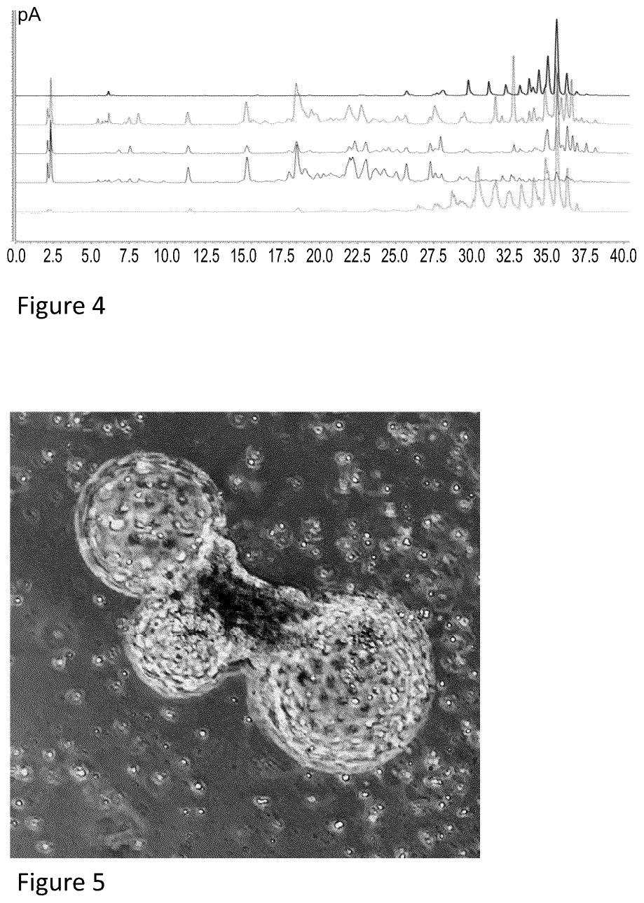

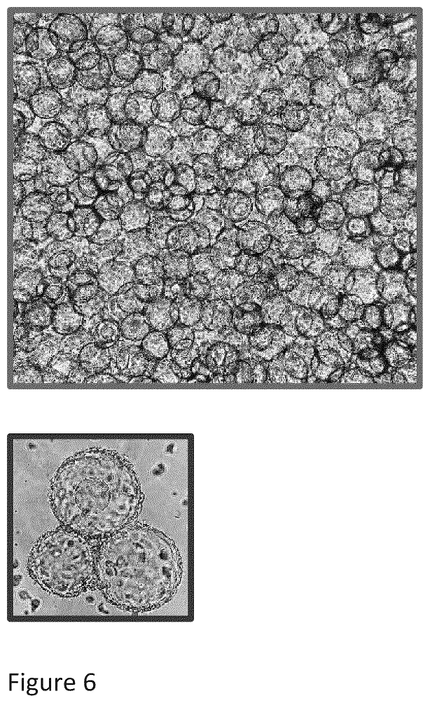

Following the isolation and expansion of mammary epithelial cells, the cells are suspended in a nutrient medium and inoculated into a culture apparatus that has been pre-coated with a mixture of extracellular matrix proteins, such as collagen, laminin, and/or fibronectin. The cell culture apparatus may be any design that allows for the compartmentalized absorption of nutrients and secretion of product from a polarized, confluent, epithelial monolayer. Examples include hollow fiber and micro-structured scaffold bioreactors (see, e.g., FIGS. 3 and 4, of WO20211142241). Alternatives include other methods of 3-dimensional tissue culture, such as the preparation of decellularized mammary gland as a scaffold, repopulated with stem cells to produce a functional organ in vitro, or collection of milk from the lumen of mammary epithelial cell organoids or “mammospheres” grown either in a hydrogel matrix or in suspension.

The apparatus includes sealed housing that maintains a temperature of about 37° C. in a humidified atmosphere of about 5% CO₂. Glucose uptake is monitored to evaluate the growth of the culture as the cells proliferate within the bioreactor. Stabilization of glucose consumption indicates that the cells have reached a confluent, contact-inhibited state. The integrity of the monolayer is ensured using transepithelial electrical resistance. Sensors monitor concentrations of dissolved O₂ and CO₂ in the media at multiple locations. A computerized pump circulates media through the bioreactor at a rate that balances the delivery of nutrients with the removal of metabolic waste such as ammonia and lactate.

Media can be recycled through the system after removal of waste using Lactate Supplementation and Adaptation technology (Freund et al. 20\SIntJMol Sci. 19 (2)) or by passing through a chamber of packed zeolite.

The medium can be supplemented with the agents described herein simultaneously or sequentially; or the medium can be replaced altogether.

According to a specific embodiment, cells can be cultured in suspension or in 2 dimensions (2D).

According to a specific embodiment, cells can be cultured under 3 dimensional (3D) conditions.

According to a specific embodiment, cells can be cultured under adherent conditions.

The Examples section below provides embodiments for 2D and 3D culturing as a monolayer or in suspension.

According to a specific embodiment, cells can be cultured directly on bioreactor membranes.

Harvesting of the lipid/milk components, also referred to herein as “secretome” is done following a predetermined time in culture starting from 12 h following induction.

According to an aspect of the invention, contemplated herein is a composition comprising cells obtainable as described herein or a secretome (e.g., complete unfractionated conditioned medium) or a fraction of said cells or the secretome (e.g., lipid fraction, protein fraction, carbohydrate fraction or combination of same).

Such a composition can find use in the food or pharma industry such as described hereinbelow.

It will be appreciated that since the cells are enriched with lipids (TGs) and especially lipid droplets, as determined by lipid staining e.g., Nile red, this fraction can be extracted from lysed cells.

Alternatively or additionally, the lipid droplets can be obtained from the medium to which they are secreted, typically in the form of fat globules FGs.

As used herein “a lipid droplets” refers to organelles found in most mammalian cells, as well as various plant tissues and yeast. They are composed of a core of neutral lipids surrounded by a membrane monolayer of phospholipids and cholesterol (i.e., micellar structure) into which specific proteins are embedded.

The core lipid core is mainly composed of triacylglycerol or cholesterol esters.

According to a specific embodiment, the size range of lipid droplets can be 200 nm to 15 μm, e.g., 200 nm to 1 μm, 200-800 nm, 200 nm-5 μm, 200 nm-5 μm.

According to a specific embodiment, the isolation of lipid droplets relies upon the buoyant density of lipid droplets, which is <1 g/cm³. Two low-speed centrifugation steps and a single ultracentrifugation step using a discontinuous density gradient will collect >95% of the lipid droplets from a cell lysate. More specifically, nuclei are removed by low-speed centrifugation (e.g., 800-2000× g) and the density of the post-nuclear supernatant is adjusted with sucrose prior to flotation of the lipid droplets through a single discontinuous sucrose gradient. The lipid droplet fraction is characterized by immunoblotting of component proteins. Details on isolation of lipid droplets from cells or media are available in Brasacmle Curr Protoc Cell Biol. 2016 Sep. 1; 72:3. 15.1-3.15.13, which is further summarized below.

The isolation of lipid droplets by centrifugation is a relatively simple procedure. A very low protein-to-lipid ratio renders lipid droplets more buoyant than all other subcellular structures; lipid droplets can be separated from more dense subcellular compartments using discontinuous density gradients. The following should be taken into account: method of cell disruption to keep the droplets intact, particularly when working with cells containing very large lipid droplets. Hence according to a specific embodiment, mechanical disruption of the cells is performed the presence of liquid nitrogen, and a Teflon pestle homogenizer. Gentle disruption of cells is required to preserve lipid droplet structure. Gentle homogenization using a hand-operated homogenizer with a loose-fitting.

The buoyant density of lipid droplets facilitates flotation during centrifugation of any aqueous medium. The use of solutions lacking electrolytes may reduce the aggregation of lipid droplets with other subcellular organelles; the use of hypotonic solutions does not compromise the integrity of lipid droplets because they have no aqueous compartment. Since segments of endoplasmic reticulum and numerous mitochondria are often closely apposed to the droplets in intact cells, isolated lipid droplet fractions contain low levels of these membranes. Contamination of lipid droplets with these membranes can be minimized by layering density-adjusted cell lysates beneath one or two layers of decreasing density in a discontinuous gradient prior to centrifugation. The movement of the lipid droplets upwards through the layers of the gradient reduces the adherence of contaminant membranes to the droplets and resolves the droplets from soluble proteins. Further removal of contaminating membranes may be enhanced by flotation of the isolated lipid droplets through additional discontinuous density gradients, which may include solutions containing a low concentration of glycerol, 100 mM to 1 M sodium chloride, or 100 mM sodium carbonate, pH 11.5 (Fujiki et al., 1982). The use of sodium carbonate wash solutions, however, may remove loosely adherent proteins that normally localize to the outer membrane monolayer of lipid droplets, and may denature the proteins. Swinging-bucket rotors are recommended to band the floating lipid droplets compactly at the top of the tube; rotors should be allowed to coast to a stop through the final deceleration to minimize disruption of the lipid droplet layer.

Following centrifugation, lipid droplets will appear as a milky layer at the top of the tube. Gentle homogenization with fewer strokes and slower insertion of the pestle will reduce disruption of the lipid droplets. There will be a pellet containing dense membranes at the bottom of the tube and one or two translucent bands of membranes at the interfaces of the density phases.

According to a specific embodiment collection of the majority of the lipid droplet fraction in a small volume is by using a Beckman tube slicer in combination with thin-walled polyallomer or polycarbonate tubes. The tube is inserted into the tube slicer to position the cutting blade several millimeters below the lipid droplet layer. The steady and firm insertion of the blade through the tube isolates the lipid droplet layer in a small volume of the top gradient solution in the sliced top portion of the tube. Rubber rings within the tube slicer form a seal with the blade, keeping the top solution from leaking out. Use of a tube slicer facilitates the most efficient collection of the lipid droplets in a minimal volume, and it permits rinsing of the surfaces of the blade and tube with additional solution to collect the fraction more completely. A pipetting device or a syringe with a wide-bore needle can be used in other embodiments.

According to a specific embodiment, collection from media is also envisaged and can be done using a similar procedure: media is concentrated using a speed-vac centrifuge, and the lipids are separated from the concentrated media using centrifugation in gradient. Alternatively, an unfractionated media is collected.

Regardless of the method employed once the droplets are at hand, according to some embodiments, they can be employed in a process which generates fat globules.

Thus, according to an aspect of the invention there is provided a method of producing synthetic fat globules, the method comprising:
(a) isolating lipid droplets from cells in culture (such as described above);
(b) encapsulating said droplets with a lipid bilayer, thereby producing synthetic fat globules.

As used herein “synthetic” refers to fat globules which are manmade involving a synthetic procedure, rather than mere isolation. Specifically, the core micellar structure is heterologous to the bilayer coating it. Since the FGs are synthetic they are also referred to as FG-like structures, or non-naturally occurring FGs.

As used herein “a Fat Globule” abbreviated as “FG”, refer to a bilayer structure which coats a micellar structure having a lipid core content.

A “milk fat globule” is composed of a core of triglycerides, cholesterol, and retinol esters that is coated by a biological membrane, i.e., the milk fat globule membrane (MFGM) which is structured as a tri-layer of polar lipids (phospholipids, sphingolipids), cholesterol, and proteins (glycoproteins and enzymes). The MFGM is found in milk.

According to a specific embodiment, the cells from which the lipid droplets are isolated are mammalian cells.

According to a specific embodiment, the cells are MECs, such as described above.

According to a specific embodiment, the cells are epithelial stem cells, e.g., mammalian (as mentioned above).

The cells can be freshly isolated, primary culture or a cell line.

Encapsulation of lipid droplets in a lipid delayer can be done using methods which are known to the skilled artisan.

According to a specific embodiment, the lipid bilayer is of a cell membrane.

According to a specific embodiment, the lipid droplet is of a different source than the cell membrane.

According to a specific embodiment, at least one of the lipid droplet and the cell membrane is from MECs.

According to a specific embodiment, both the lipid droplet and the cell membrane is from MECs.

According to a specific embodiment, the lipid droplet is from MECs.

According to a specific embodiment, the cell membrane is from MECs.

The membranal and lipid droplets fraction is used to generate thin lipid film layers as a step to produce lipid micelles (H. Zhang, 2017; W. Zhang et al., 2018), and as a first step in MFGL (MFG Like) structure production. The mixture of membranes and lipids in the thin lipid layer results in liposomal like structures, containing both membranal and lipid droplet elements.

Another way to produce the MFGLs is by 30 cycles of 2 min heating-3 min cooling, with vortex while cooling, this method also provides MFGLs.

To further reduce the size of the MFGL and to render the population more homogenous, the MFGLs are homogenized by sonication (Mendez & Banerjee, 2017) or extrusion (Gven et al., 2009). Both methods result in homogenization of MFGLs to the size of <20 μM. Homogenizing the final product increases its stability, and increases the membrane fraction by increasing the (membrane/lipid droplet size) ratio.

An exemplary method for lipid extraction is as follows:

Dried the sample (cells, media) in a new weighed glass tubes using rotary evaporator. A dried is milled to powder in the tube and weighed. Ethanol is added to the dry milled sample. Th combination is vortexed and extracted two times (e.g., for 20-200 min at 45-800° C. with interval of 2-40 min at RT and vortex). Ethanol volume is calculated by dilution of dried milled sample (mg), e.g., to 10 mg. At the end of extraction, the sample is centrifuged during 30 min at 4-250° C. The organic phase is pipetted using a new glass pipet to new glass tube. The material is washed. Then Hexane is added to ethanol in the tube according to calculation to 1 volume of Ethanol add 1-6 volumes of Hexane and vortexed for 5-60 sec. After 20 min at RT, centrifugation takes plance e.g., 400-5000xG during 30 min at 4-250° C. The clear liquid is pipetted using glass pipet to a new glass tube and the residue is washed with a small portion of hexane, centrifuged and the solvent is pipetted to the previously tube with Hexane-Ethanol mixture. The extracted Hexane-Ethanol mixture is evaporated using rotary evaporator. Oil is dissolved in small volume of Hexane and pipetted to a new weighed glass vials using glass pipet and evaporated using rotary evaporator.

According to an embodiment, the droplets and membranes are isolated and subjected to extrusion and/or sonication such that particles of a size range of 100 nm to 10 μm are formed comprising a coating cell membrane and a micellar inner core, which corresponds to the lipid droplet.

According to a specific embodiment, the membrane bilayer comprises peptides, proteins and/or carbohydrates.

The structure can be different than natural FGs by the protein content, for instance. For example, reduced protein content or a different protein or peptide profile in the coating bilayer structure.

Alternatively, the lipid bilayer is a synthetic membrane, such as a liposome having at least one layer of a phospholipid bilayer membrane encapsulating a hydrophobic core.

For example, pure phospholipids from egg yolk or soybeans are dissolved, and are gently evaporated in a rotary evaporator to generate thin layer of phospholipids. After full evaporation of solvent, pre-heated lipids (to 50° C.) are added to form micellar bodies, and the procedure is enhanced using vortex and sonication. At last step the micellar bodies are homogenized using an Avanti extruder.

Additional examples for producing FGs from synthetic bilayers are provided in the references infra.

Zhang, H. (2017). Thin-film hydration followed by extrusion method for liposome preparation. In Methods in Molecular Biology (Vol. 1522, pp. 17-22). Humana Press Inc. www (dot) doi (dot) org/10.1007/978-1-4939-6591-5_2

Zhang, W., Li, C., Jin, Y., Liu, X., Wang, Z., Shaw, J. P., Baguley, B. C., Wu, Z., & Liu, J. (2018). Multiseed liposomal drug delivery system using micelle gradient as driving force to improve amphiphilic drug retention and its anti-tumor efficacy. Drug Delivery, 25 (1), 611-622. www (dot) doi (dot) org/10.1080/10717544.2018.1440669

Bruce et al. Ann. Rev. Physiol. 1984.46:417-33

Keenan et al. Analytical Biochemistry 177,194-1% (1989) (isolation of plasma membranes from mammary glands).

Of note FGs are smaller than cells. They are devoid of intact cells or cell organelles.

Once the FGs are obtained they can be implemented in various nutritional products.

Thus, according to an aspect of the invention there is provided a composition comprising a synthetic fat globule structure.

According to a specific embodiment, the composition is obtainable according to the method as described herein.

According to a specific embodiment, the composition is a food or a beverage.

Also provided is a method of producing a nutritional product, the method comprising combining food or beverage with the composition as described herein

The composition comprising the secretome or portions thereof, MFGs, cells,can be used per se, or combined with components of the food, feed or beverage industry, pharmaceutical industry for human or veterinary use.

Thus, according to an aspect of the invention there is provided a nutritional composition which may be food or feed comprising the FGs, cells, secretome or portions thereof (e.g., lipid droplets).

The food can be vegan, vegetarian, dairy or may comprise meat.

As used herein “food” refers to both food (human consumption), feed (animal consumption), liquid (beverage), solid or semi-solid.

Thus, according to an aspect of the invention, there is provided a method of producing food or feed comprising combining the composition comprising the cells, lipid droplets, secretome or portions thereof or FGs in a food production process.

The process of producing food may include any of rising, kneading, extruding, molding, shaping, cooking, boiling, broiling, baking, frying and any combination of same.

Also provided is a method of providing nutrition to a subject in need thereof. The method comprising providing the subject with a foodstuff as described herein.

According to a specific embodiment, the subject is at risk of nutritional deficiency.

According to a specific embodiment, the subject is a healthy subject (e.g., not suffering from a disease associated with nutrition/absorption).

According to a specific embodiment the food is an “infant formula” as used herein refers to a nutritional composition intended for infants and as defined in Codex Alimentarius, (Codex STAN 72-1981) and Infant Specialties (incl. Food for Special Medical Purpose) as defined in Codex Alimentarius, (Codex STAN 72-1981). It also refers to a foodstuff intended for particular nutritional use by infants during the first months of life and satisfying by itself the nutritional requirements of this category of person (Article 2 (c) of the European Commission Directive 91/321/EEC 2006/141/EC of 22 Dec. 2006 on infant formulae and follow-on formulae). The infant formulas encompass the starter infant formulas and the follow-up or follow-on formulas. Generally, a starter formula is for infants from birth as breast-milk substitute, and a follow-up or follow-on formula from the 6th month onwards.

Other contemplated products include, but are not limited to, milk, butter, cream, cheese, ice cream, yogurt. These products can be products obtained in culture, e.g., cultured milk; or natural milk to which a component obtained at least in part in culture e.g., lipids or FGs.

In some embodiments, the lipid droplets, e.g., FGs, may be added to the nutritional composition by replacing an equivalent amount of the rest of the overall fat blend normally present in the nutritional composition. In some embodiments, a certain amount of oil used as a fat source, that does not contain the milk fat globules described herein may be substituted with the lipid droplets, e.g., FGs. In yet another embodiment, the nutritional composition may be supplemented with the lipid droplets, e.g., GFs. In some embodiments, the lipid droplets, e.g., FGs, may be the sole fat source added to the nutritional composition.

In some embodiments, the nutritional composition described herein comprises a fat source. The lipid droplets, e.g., FGs described herein may be the sole fat source or may be used in combination with any other suitable fat or lipid source for the nutritional composition as known in the art. Appropriate fat sources include, but are not limited to, animal sources, e.g., milk fat, butter, butter fat, egg yolk lipid; marine sources, such as fish oils, marine oils, single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, wheat germ oil; medium chain triglyceride oils and emulsions and esters of fatty acids; and any combinations thereof.

The disclosed nutritional composition described herein can, in some embodiments, also comprise a source of prebiotics. The term “prebiotic” as used herein refers to indigestible food ingredients which exert health benefits upon the host. Such health benefits may include, but are not limited to, selective stimulation of the growth and/or activity of one or a limited number of beneficial gut bacteria, stimulation of the growth and/or activity of ingested probiotic microorganisms, selective reduction in gut pathogens, and favorable influence on gut short chain fatty acid profile. Such prebiotics may be naturally-occurring, synthetic, or developed through the genetic manipulation of organisms and/or plants, whether such new source is now known or developed later. Prebiotics useful in the present disclosure may include oligosaccharides, polysaccharides, and other prebiotics that contain fructose, xylose, soya, galactose, glucose and mannose.

More specifically, prebiotics useful in the present disclosure may include polydextrose, polydextrose powder, lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, galacto-oligosaccharide, and gentio-oligosaccharides. In one preferred embodiment, the prebiotic comprises galacto-oligosaccharide, polydextrose, or mixtures thereof.

The disclosed composition described herein can, in some embodiments, also comprise a source of probiotic. The term “probiotic” means a microorganism that exerts beneficial effects on the health of the host. Any probiotic known in the art may be acceptable in this embodiment. In a particular embodiment, the probiotic may be selected from any Lactobacillus species, Lactobacillus rhamnosus GG (ATCC number 53103), Bifidobacterium species, Bifidobacterium longum BB536 (BL999, ATCC: BAA-999), Bifidobacterium longum AH1206 (NCIMB: 41382), Bifidobacterium breve AH1205 (NCIMB: 41387), Bifidobacterium infantis 35624 (NCIMB: 41003), and Bifidobacterium animalis subsp. lactis BB-12 (DSM No. 10140) or any combination thereof.

The nutritional compositions of the present disclosure may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The nutritional compositions of the disclosure may provide minimal, partial or total nutritional support. The compositions may be nutritional supplements or meal replacements. The compositions may, but need not, be nutritionally complete. In an embodiment, the nutritional composition of the disclosure is nutritionally complete and contains suitable types and amounts of lipid, carbohydrate, protein, vitamins and minerals.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Primary Culture

A primary culture of MEC was isolated from mammary biopsies. Briefly, udder tissue was collected from lactating cows in a commercial slaughterhouse and immediately submerged in ice-cold growth medium, DMEM-F12, with 1000 U/ml penicillin, 1 mg/ml streptomycin, 2.5 μg/ml amphotericin B mixture, and 0.02 mg/ml heparin supplementation to prevent cell clotting after digestion. Study protocols followed the regulations of the Israeli Ministry of Health.

After transfer to the laboratory, tissue was minced and digested by shaking in a growth medium supplemented with collagenase (1 mg/ml), hyaluronidase (1 mg/ml) and 0.02 mg/ml heparin, at 100 rpm for 3 h at 37° C. After incubation, the suspension was filtered through a metal mesh (250 μm) and the filtrate was centrifuged at 350 g for 5 min. The sediment was treated with trypsin-EDTA and 0.04% (w/v) DNase. The cells were then washed with growth medium supplemented with heparin and treated with DNase alone, filtered through a 100-μm cell strainer (BD Falcon, Bedford, MA) and washed with the growth medium.

Cells were grown in plastic culture dishes with DMEM/F12 supplemented with 10% (w/v) FBS, 100 U/ml penicillin, 100 μg/ml streptomycin, 0.25 μg/ml amphotericin B supplemented with insulin (1 μg/ml), hydrocortisone (0.5 μg/ml) and prolactin (1 μg/ml).

### Induction Medium:

DMEM/F12 supplemented with 10% (w/v) FBS, 100 U/ml penicillin, 100 μg/ml streptomycin, 0.25 μg/ml amphotericin B supplemented with insulin (1 μg/ml), hydrocortisone (0.5 μg/ml) and prolactin (1 μg/ml).

### Treatments

1. Oleic acid treatment: Oleic acid was added to the induction medium at 360-1500 mM final concentration. Treatment was carried for 1 up to 7 days.

2. LXRa treatment: Liver X Receptor agonist (LXRa), is a known activator of the mouse LXR, and suggested to act similarly on the human and bovine orthologs (NR1H3). LXRa was added into the media with or without Oleic acid. Two LXR agonists were tested: GW3965 (LXR1, SIGMA) and T0901317 (LXR2, SIGMA), each at a concentration of luM/ml for at least overnight (ON) incubation. The OA was added after cells reached the confluency of at least 80%.

### Lipid Extraction and Analysis

After treatment, cells were harvested with trypsin (0.05%), washed with 0.9% (w/v) NaCl and stored at −20° C. until lipid extraction.

Total lipids were extracted from the intact cells by NP-40 and from media by cold-extraction procedure developed by Folch et al. with few adaptations. Briefly, a 5-ml aliquot of methanol: chloroform solution (2:1, v/v) was added to each sample. After incubation at room temperature, 1 ml of double-distilled water was added. After overnight incubation at 4° C., the upper phase was discarded, and the lower phase was filtered through a Pasteur pipette with glass wool. Samples were then dried under a nitrogen stream at 65° C., diluted in 100 μl 0.5% NP-40. Of note, lipid metabolites are measured by Cobas measurement, which is a parameter for lipid activity but measures also glycerol, the plate assay on the other hand measures TGs specifically while eliminating the glycerol background.

After treatment, medium was collected and centrifuged at 500 g for 10 min. The supernatant was transferred to a fresh tube and kept at 4° C. until analysis. Medium triglyceride content was quantified with a Triglyceride Quantification Kit (lipase assay Abcam, Cambridge, UK). Quantification was performed against external standard curves and expressed as μg/10∧6 live cells. Live cell number was determined with a hemocytometer after Trypan blue staining.

### Lipid Droplet Staining

Cells grown on glass cover slips were rinsed three times with PBS and fixed with 4% paraformaldehyde in PBS for 20 min at room temperature. Then the cover slips were rinsed four times with PBS and stained with Nile red (200 nM, Sigma) for 15 min. Cover slips were then rinsed three times with PBS and stained with DAPI (Sigma) for 5 min. Finally, cover slips were rinsed four times with PBS and mounted with fluorescent mounting medium (Dako). Fluorescence Microscopy and Lipid Droplet Size Measurements Slides were visualized with an Echo fluorescence microscope equipped with an digital camera. Lipid droplet analysis was done using ImageJ software.

### Gene Expression

### RT-PCR Protocol

Cells were harvested using the TYRPLE™ reagent and pellet was collected by centrifugation of 3 min in 300 rcf. RNA was extracted from 1×10⁶ cells using Nzytech™ total RNA isolation kit (MB13402). cDNA was prepared using SensiFast™ cDNA kit (0.5μgr RNA/20 μl reaction mix). RT-PCR was done with the following primers by using SensiFast™ RT-PCR kit with 1.25-20 ng cDNA/well.

TABLE 1

| Primer | | NCBI | | | | | |
|---|---|---|---|---|---|---|---|
| ID | Primer | Reference | Primer sequence | Tm | | Length | Product |
| Number | Name | Sequence | (5′→3′) | ° C. | GC% | (bases) | Size |
| 5 | 5_bGAPDH_ | NM_ | TGGTGAAGGTCGGAGTGA | 63 | 55 | 20 | 154 |
| | F1 | 001034034.2 | AC/SEQ ID NO: 1 | | | | |
| | 6_bGAPDH | | CCGTGGGTGGAATCATACT | 62 | 55 | 20 | |
| 6 | R1 | | G/SEQ ID NO: 2 | | | | |
| | 7_bGAPDH | | CTGCCCGTTCGACAGATAG | 64 | 60 | 20 | 95 |
| 7 | F2 | | C/SEQ ID NO: 3 | | | | |
| 8 | 8_bGAPDH | | ATGCGGCCGAATCCGTTCA | 67 | 60 | 20 | |
| | R2 | | C/SEQ ID NO: 4 | | | | |
| 41 | 41_ | NM_ | CCCATGGAGACCATCAAG/ | 58 | 55 | 18 | 167 |
| | bSLC25A1_F1 | 174656.2 | SEQ ID NO: 5 | | | | |
| 42 | 42_ | | ATGCCCTGATTGGATCCC/ | 60 | 55 | 18 | |
| | bSLC25A1_R1 | | SEQ ID NO: 6 | | | | |
| 43 | 43_ | | TGGGTCTGGGAAAGCCAAA | 64 | 55 | 20 | 114 |
| | bSLC25A1_F2 | | C/SEQ ID NO: 7 | | | | |
| 44 | 44_ | | AGCTGCGTCTTCACGTACTC | 64 | 55 | 20 | |
| | bSLC25A1_R2 | | /SEQ ID NO: 8 | | | | |
| 49 | 49_bFASN_ | NM_ | GAAGGACCTGTCCCGGTTT | 64 | 60 | 20 | 189 |
| | F1 | 001012669.1 | G/SEQ ID NO: 9 | | | | |
| 50 | 50_bFASN_ | | GAAGCCTCAGAGCCACTCA | 64 | 60 | 20 | |
| | R1 | | C/SEQ ID NO: 10 | | | | |
| 51 | 51_bFASN_ | | GTTTGACGCTTCCTTCTTCG | 61 | 50 | 20 | 125 |
| | F2 | | /SEQ ID NO: 11 | | | | |
| 52 | 52_bFASN_ | | GAATGGAAGCTGGGTTGAT | 60 | 50 | 20 | |
| | R2 | | G/SEQ ID NO: 12 | | | | |
| 53 | 53_bSQLE_ | NM_ | GCAGCCAGGTGGTTATTAT | 60 | 50 | 20 | 140 |
| | F1 | 001098061.1 | G/SEQ ID NO: 13 | | | | |
| 54 | 54_bSQLE_ | | CTGACAGAGGGAAAGGAA | 59 | 50 | 20 | |
| | R1 | | TC/SEQ ID NO: 14 | | | | |
| 55 | 55_bSQLE_ | | GGCTGCAGTGCTTTCCAGA | 65 | 60 | 20 | 158 |
| | F2 | | G/SEQ ID NO: 15 | | | | |
| 56 | 56_bSQLE_ | | CCTGGGCATCAATACCTTCC | 62 | 55 | 20 | |
| | R2 | | /SEQ ID NO: 16 | | | | |
| 57 | 57_bNR1H3_ | NM_ | ACGTCTGCGATTGAGGTGA | 63 | 50 | 20 | 116 |
| | F1 | 001014861.1 | T/SEQ ID NO: 17 | | | | |
| 58 | 58_bNR1H3_ | | CCTGCTTTGGCAAAGTCTTC | 61 | 50 | 20 | |
| | R1 | | /SEQ ID NO: 18 | | | | |
| 59 | 59_bNR1H3_ | | ATAACCGGGAAGACTTTGC | 62 | 50 | 20 | 141 |
| | F2 | | C/SEQ ID NO: 19 | | | | |
| 60 | 60_bNR1H3_ | | CTGCAGAGAAGATGCTGAT | 60 | 50 | 20 | |
| | R2 | | G/SEQ ID NO: 20 | | | | |

### Example 1

### Lxr Agonist Affected Gene Expression of Lipid Synthesis and Metabolism Genes

Activation of lipid metabolism was tested by RT-PCT for LXR downstream genes: SREBP1, FASN, SLC25A1 and SQLE. Over-expression of x5 fold was observed for SREBP1, and x2 for FASN, while for SLC25A1 and SQLE there was no change in gene expression. These results suggest that the LXR agonist activates the LXR receptor, NR1H3. Signal reduction along the pathway implies signal conversion from gene expression regulation to metabolic feedback loops. Although lipid droplets were observed following OA incubation, there was no effect of OA on LXR dependent gene expression of lipid metabolism, while LXR agonist on LXR pathway activation was OA-independent.

### Example 2

### Lxr Agonists Enhance Lipid Metabolism and Ameliorate the Negative Effect of Oleic Acid on Tissue Culture

Oleic acid treatment, when used in high concentrations of above 300 μM or for long periods of longer than one day, has a negative effect on cells viability. This is probably due to the accumulation of lipid bodies in the cytoplasm to abnormally high concentrations, leading to losing cell functionality, cytotoxic effect, and finally, cell death (FIG. 2). Indeed for cells treated with oleic acid, intracellular accumulation of lipid bodies was observed (white-yellowish bodies in the bright field). The analysis was done for the presence of 360 μM OA or 1100 μM OA with or without LXRa (TO901317). Another observation was intensive cell death, suggesting a correlation between lipid accumulation, cytotoxic effect, and cell death.

One of the major reasons for lipid accumulation is the lack of lipid bodies released from the cells to the media.

The addition of 1 μM/ml LXR resulted in higher lipid metabolism, but also in lipids release to the medium and cells survival. Interestingly, although under the effect of LXRa cells release lipids to the medium, the total TGs measured for IM cells are similar or higher for LXR-OA treated cells, when compared to OA only (FIG. 3). This suggests that lipids are more equally spread in the cells, and therefore less cytotoxic, and that lipid production is higher for LXRa treated cells.

The additive effect of LXR agonist on lipids production measured intracellularly was observed mainly when using oleic acid at concentrations of 360 μM or below. For higher oleic acid concentrations there was less or no effect in cellular TGs detected by plate assay, however; lipid metabolites levels in the media were higher for LXR treated samples.

More specifically, LXRa treatment (1 μl/ml) didn't reduce the total TGs observed in the cells for any concentration of OA treatment. For OA concentrations higher than 360 μM the effect of LXR agonist was not observed intracellularly, but only on media metabolites. The LXRa treated group showed 60% higher intracellular TGs levels for the 360 μM OA treatment. IM control: induction media control. GM control: growth media control. OA control: Oleic acid loaded on BSA without NaOH addition.

### Example 3

### Preparation of Milk Fat Globules Like (MFGLs) Structures

MEC cultures—as above.

Lipid synthesis inductions—with LXRa and OA as above.

Lipid extraction and analysis—as above.

### Membranal and Lipid Separation

Cleaning the fraction of membranes and lipids from nuclei and mitochondrial derbies is done by fraction collection of the upper fraction of lysed cell pellet after a long centrifugation, or by collection of the lipophilic fraction using column based isolation, by gentle grinding of the cells, incubation of 10 min on ice and strong centrifugation of 16,000×g. The supernatant was collected for further gradient centrifugation as described above (Eski et al., 2020).

### Lipids and Lipid Droplet Staining

For lipid staining, the collected mass was mixed with 10 μl Nile red (0.1% w/v in ethanol). After 1 h of incubation, the sample was mixed thoroughly with an equal volume of 0.5% low melting point agarose in 10 mM di-sodium hydrogen phosphate buffer, pH 6.8, then 20 μl of the stained sample was placed on a glass cavity slide Fluorescence microscopy and lipid droplet size measurements slides were visualized with an Echo fluorescence microscope equipped with a digital camera using the RFP channel. Lipid droplet analysis was done using ImageJ software.

### Generation of Lipid Film Layers

In order to create lipid micelles, a step involving using a combination of membranal and lipid droplets fractions is included to produce thin layers of lipid film. This process, described by H. Zhang in 2017 and W. Zhang et al. in 2018, results in a heterogeneous product that contains both membranal and lipid droplet elements in the form of liposomal-like structures. This step serves as the initial stage in the production of MFGL structures. Another way to produce the MFGLs is by 30 heating—cooling cycles of 2 min, with 3 min vortex while cooling, this method also provides heterogeneous MFGLs. At the size of 200 nM to 10 μM.

The MFGLs are homogenized by sonication (Mendez & Banerjee, 2017) or extrusion (Gven et al., 2009). Both methods result in homogenization of MFGLs to the size of <20 μM, the aim of homogenizing the final product is to increase its stability, and to increase the membrane fraction by increasing the (membrane/lipid droplet size) ratio.

### Example 4

The Effect of the Transgenes LXR and PRLR in Human Mammary Epithelial Cells from Mammary Gland on TAGs Metabolism

The human LXR gene (hNR1H3 [NM_001251935.2]) and the human PRLR gene (hPRLR [NM_000949.7]) were inserted into a plasmid that conferred resistance to Puromycin. Mammary epithelial cells were transfected with the plasmid using PolyJet transfection reagent, with a concentration of lug of DNA per 1X10⁶ cells in suspension. The cells were then seeded into a 6-well plate and incubated for 10 minutes at room temperature before adding 2 ml of growth media to each well. The plate was then moved to a 37C incubator with 5% CO₂. The following day, the growth media was replaced with fresh media containing lug/ml of puromycin and the cells were cultured for 10 days. Lipid profile was obtained using HPLC-CAD, and triglycerides were quantified using Abnova's ELISA kit.

Table 2 below shows the synergic effect of LXR and PRLR on TAGs metabolism in human mammary epithelial cells from mammary gland. For the human cell line a significant increase in TAGs metabolism was observed for the transgenic cell lines, compared to the naïve cells. Although LXR provided higher TAGs content compared to WT and PRLR, the combination of LXR and PRLR provided a synergic effect in TAGs production.

TABLE 2

| ID | Transgene | TAGs ug/1M Cells |
|---|---|---|
| 1 | WT | 7 |
| 2 | PRLR | 34 |
| 3 | LXR | 44 |
| 4 | LXR + PRLR | 60.3 |

### Example 5

### Growth of Mammary Epithelial Cells as Spheroids in Suspension

### Spheroids Formation in Aggrewell

Mammary epithelial cells spheroids were generated in large quantities using the Aggrewell plates platform (Stemcell technologies). Each well in these plates contains a standardized array of microwells, 400 μm or 800 μm in size. Plates were pre-coated with an anti-adherence solution (Stemcells technologies). After coating, 50, 100, 150, 200, 500, or 900 cells per microwell were seeded following Stemcells technologies standard protocol. Stable spheroids were observed 1-3 days after seeding. Spheroids were collected from the Aggerwell and transferred to suspension culture.

### Example 6

### Growth of Mammary Epithelial Cells on Beads in Suspension

Cytodex I microcarrier beads were first activated by hydration in Ca2+ and Mg2+ free PBS at a density of 225 mg microbeads per 50 to 100 mL/g of PBS, and then incubated at room temperature over night. Before seeding, carriers were washed twice according to the manufacturer's preparation protocol; and centrifuged for 10 min 1200 rcf. MECs (as above) were seeded on the Cytodex I carriers within spinner flask of 500 ml for 2 hr. Thereafter. the suspension was transferred to a 1.6 L bioreactor; with an agitation speeding rate of 50 rpm. The final seeding density in the bioreactor is 0.1-0.5 mg microbeads per ml.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### (Other Reference are Cited in the Document)

Eski, S. E., Dubois, C., & Singh, S. P. (2020). Nuclei isolation from whole tissue using a detergent and enzyme-free method. Journal of Visualized Experiments, 2020 (160), 1-10. www (dot) doi (dot) org/10.3791/61471
Gven, A., Ortiz, M., Constanti, M., & O'Sullivan, C. K. (2009). Rapid and efficient method for the size separation of homogeneous fluorescein-encapsulating liposomes. Journal of Liposome Research, 19 (2), 148-154. www (dot) doi (dot) org/10.1080/08982100802674419
Hernell, O., Timby, N., Domellof, M., & Lönnerdal, B. (2016). Clinical Benefits of Milk Fat Globule Membranes for Infants and Children. Journal of Pediatrics, 173, S60-S65. www (dot) doi (dot) org/10.1016/j.jpeds.2016.02.077
Mendez, R., & Banerjee, S. (2017). Sonication-based basic protocol for liposome synthesis. In Methods in Molecular Biology (Vol. 1609, pp. 255-260). Humana Press Inc. www (dot) doi (dot) org/10.1007/978-1-4939-6996-8_21
Timby, N., Domellof, M., Lönnerdal, B., & Hernell, O. (2017). Supplementation of infant formula with bovine milk fat globule membranes. In Advances in Nutrition (Vol. 8, Issue 2, pp. 351-355). American Society for Nutrition. www (dot) doi (dot) org/10.3945/an.116.014142
Zhang, H. (2017). Thin-film hydration followed by extrusion method for liposome preparation. In Methods in Molecular Biology (Vol. 1522, pp. 17-22). Humana Press Inc. www (dot) doi (dot) org/10.1007/978-1-4939-6591-5_2
Zhang, W., Li, C., Jin, Y., Liu, X., Wang, Z., Shaw, J. P., Baguley, B. C., Wu, Z., & Liu, J. (2018). Multiseed liposomal drug delivery system using micelle gradient as driving force to improve amphiphilic drug retention and its anti-tumor efficacy. Drug Delivery, 25 (1), 611-622. www (dot) doi (dot) org/10.1080/10717544.2018.1440669

1. A method of increasing lipid synthesis or accumulation in mammary epithelial cells (MECs) culture and optionally secretion therefrom, the method comprising contacting the MECs with an LXR agonist in the presence of a fatty acid to thereby increase lipid synthesis or accumulation and optionally secretion of lipids.

2. The method of claim 1, wherein said fatty acid is selected from the group consisting of oleic acid, palmitic acid, linoleic acid, arachidonic acid, eicosapentaenoic acid and docosahexaenoic acid or wherein said fatty acid is selected from the group consisting of oleic acid and palmitic acid.

3-4. (canceled)

5. The method of claim 1, wherein said contacting is in the presence of at least one of a phenolic composition, insulin, hydrocortisone, prolactin and 8-hydroxybutirate (BHBA).

6. The method of claim 1, further comprising:
harvesting lipids from said MECs or from a medium of said culture of said MECs.

7. (canceled)

8. The method of claim 1, wherein:
said LXR agonist is GW3965 or T0901317;
said LXR agonist is 9-cis-13,14-dihydroretinoic acid; and/or
said LXR agonist is at least one nucleic acid agent which upregulates LXR, an activator and/or effector thereof, optionally wherein said LXR agonist is at least one nucleic acid agent which upregulates LXR, an activator and/or effector thereof, optionally said nucleic acid agent encodes LXR, optionally said at least one nucleic acid agent encodes LXR and PRLR, optionally wherein said at least one nucleic acid agent introduces a nucleic acid alteration in a genomic sequence of said LXR, an activator and/or effector thereof.

9-17. (canceled)

18. The method of claim 8, wherein said nucleic acid alteration renders an LXR response element (LXRE) constitutively active.

19. The method of claim 8, wherein said nucleic acid alteration renders an LXR response element (LXRE) inducible.

20. The method of claim 5, wherein said culture comprises said LXR agonist, fatty acid and said phenolic composition.

21. The method of claim 5, wherein said phenolic composition is selected from the group consisting of flavonol, flavanol, flavone, flavanone and anthocyanidin.

22. The method of claim 5, wherein said phenolic composition is selected from the group consisting of gallic acid or myricetin or derivative thereof.

23. The method of claim 1, effected in the presence of albumin, optionally wherein said albumin is bovine serum albumin (BSA), optionally wherein said albumin is BSA or plant albumin.

24-25. (canceled)

26. The method of claim 1, wherein said MECs are human or bovine MECs.

27. A composition comprising cells obtainable according to the method of claim 1 or secretome or a fraction of said cells or said secretome.

28. A method of producing synthetic fat globules, the method comprising:
(a) isolating lipid droplets from cells in culture;
(b) encapsulating said droplets with a lipid bilayer, thereby producing synthetic fat globules.

29. The method of claim 28, wherein said cells are mammary epithelial cells (MECs).

30. The method of claim 28, wherein said a lipid bilayer is a cell membrane.

31. The method of claim 30, wherein said cell membrane is of mammary epithelial cells (MECs).

32. The method of claim 28, wherein said lipid bilayer is a synthetic membrane optionally wherein said lipid bilayer comprises peptides, optionally wherein said membrane and said lipid droplet content are from different sources, optionally wherein one source of said different sources comprises MECs, optionally wherein said source is a mammary epithelial cell (MEC).

33-37. (canceled)

38. The method of claim 28, further comprising subjecting said cells to a lipid synthesis induction protocol prior to step (a), optionally.

39-40. (canceled)

41. A composition comprising synthetic fat globules which comprise a lipid droplet core coated with a lipid bilayer, optionally wherein said synthetic fat globules are of a size range of 100 nm-15 μm.

42-43. (canceled)


SEQUENCE LISTING (20 sequences)

SEQ ID NO: 1 - DNA, 20 bp, synthetic construct
  misc_feature 1..20: note = Single strand DNA oligonucleotide
  source 1..20: mol_type = other DNA
tggtgaaggtcggagtgaac

SEQ ID NO: 2 - DNA, 20 bp, synthetic construct
  misc_feature 1..20: note = Single strand DNA oligonucleotide
  source 1..20: mol_type = other DNA
ccgtgggtggaatcatactg

SEQ ID NO: 3 - DNA, 20 bp, synthetic construct
  misc_feature 1..20: note = Single strand DNA oligonucleotide
  source 1..20: mol_type = other DNA
ctgcccgttcgacagatagc

SEQ ID NO: 4 - DNA, 20 bp, synthetic construct
  misc_feature 1..20: note = Single strand DNA oligonucleotide
  source 1..20: mol_type = other DNA
atgcggccgaatccgttcac

SEQ ID NO: 5 - DNA, 18 bp, synthetic construct
  misc_feature 1..18: note = Single strand DNA oligonucleotide
  source 1..18: mol_type = other DNA
cccatggagaccatcaag

SEQ ID NO: 6 - DNA, 18 bp, synthetic construct
  misc_feature 1..18: note = Single strand DNA oligonucleotide
  source 1..18: mol_type = other DNA
atgccctgattggatccc

SEQ ID NO: 7 - DNA, 20 bp, synthetic construct
  misc_feature 1..20: note = Single strand DNA oligonucleotide
  source 1..20: mol_type = other DNA
tgggtctgggaaagccaaac

SEQ ID NO: 8 - DNA, 20 bp, synthetic construct
  misc_feature 1..20: note = Single strand DNA oligonucleotide
  source 1..20: mol_type = other DNA
agctgcgtcttcacgtactc

SEQ ID NO: 9 - DNA, 20 bp, synthetic construct
  misc_feature 1..20: note = Single strand DNA oligonucleotide
  source 1..20: mol_type = other DNA
gaaggacctgtcccggtttg

SEQ ID NO: 10 - DNA, 20 bp, synthetic construct
  misc_feature 1..20: note = Single strand DNA oligonucleotide
  source 1..20: mol_type = other DNA
gaagcctcagagccactcac

SEQ ID NO: 11 - DNA, 20 bp, synthetic construct
  misc_feature 1..20: note = Single strand DNA oligonucleotide
  source 1..20: mol_type = other DNA
gtttgacgcttccttcttcg

SEQ ID NO: 12 - DNA, 20 bp, synthetic construct
  misc_feature 1..20: note = Single strand DNA oligonucleotide
  source 1..20: mol_type = other DNA
gaatggaagctgggttgatg

SEQ ID NO: 13 - DNA, 20 bp, synthetic construct
  misc_feature 1..20: note = Single strand DNA oligonucleotide
  source 1..20: mol_type = other DNA
gcagccaggtggttattatg

SEQ ID NO: 14 - DNA, 20 bp, synthetic construct
  misc_feature 1..20: note = Single strand DNA oligonucleotide
  source 1..20: mol_type = other DNA
ctgacagagggaaaggaatc

SEQ ID NO: 15 - DNA, 20 bp, synthetic construct
  misc_feature 1..20: note = Single strand DNA oligonucleotide
  source 1..20: mol_type = other DNA
ggctgcagtgctttccagag

SEQ ID NO: 16 - DNA, 20 bp, synthetic construct
  misc_feature 1..20: note = Single strand DNA oligonucleotide
  source 1..20: mol_type = other DNA
cctgggcatcaataccttcc

SEQ ID NO: 17 - DNA, 20 bp, synthetic construct
  misc_feature 1..20: note = Single strand DNA oligonucleotide
  source 1..20: mol_type = other DNA
acgtctgcgattgaggtgat

SEQ ID NO: 18 - DNA, 20 bp, synthetic construct
  misc_feature 1..20: note = Single strand DNA oligonucleotide
  source 1..20: mol_type = other DNA
cctgctttggcaaagtcttc

SEQ ID NO: 19 - DNA, 20 bp, synthetic construct
  misc_feature 1..20: note = Single strand DNA oligonucleotide
  source 1..20: mol_type = other DNA
ataaccgggaagactttgcc

SEQ ID NO: 20 - DNA, 20 bp, synthetic construct
  misc_feature 1..20: note = Single strand DNA oligonucleotide
  source 1..20: mol_type = other DNA
ctgcagagaagatgctgatg